(19) United States
(12) Patent Application Publication
(10) Pub. No.: US 2007/0123841 A1
(43) Pub. Date: May 31, 2007

(54) Intravenous tubing attachment

(76) Inventors: Laura S. Egan, Palisades Park, NJ (US); Marc Stipo, Branchville, NJ (US)

Correspondence Address: LITMAN LAW OFFICES, LTD., P.O. BOX 15035, CRYSTAL CITY STATION, ARLINGTON, VA, 22215, US

(21) Appl. No.: 11/604,271

(22) Filed: Nov. 27, 2006

Related U.S. Application Data
(60) Provisional application No. 60/740,684, filed on Nov. 30, 2005.

(51) Int. Cl. A61M 25/00

(52) U.S. Cl. 604/523; 604/80

(57) ABSTRACT

The intravenous tubing attachment is a device for restricting movement of a clamp mounted on intravenous tubing. The intravenous tubing attachment includes a main body portion having a pair of opposed planar surfaces, which are dimensioned and configured for receiving optional adhesive labels. A clip is formed on a proximal end of the main body portion, having a pair of arcuate jaws defining a recess for releasably engaging the intravenous tubing without restricting fluid flow through the intravenous tubing. The engagement of the intravenous tubing with the recess maintains the intravenous tubing attachment in one selectable position on the tubing and restricts movement of a clamp mounted on the intravenous tubing, preventing slippage of the clamp on the tubing.

### CROSS-REFERENCE TO RELATED APPLICATION

This application claims the benefit of U.S. Provisional Patent Application Ser. No. 60/740,684, filed Nov. 30, 2005.

### BACKGROUND OF THE INVENTION

1. Field of the Invention

The present invention relates to clips attachable to tubing, and particularly to an intravenous tubing attachment that clips onto an intravenous infusion line below the roller clamp to prevent the roller clamp from sliding down the tubing.

2. Description of the Related Art

A wide variety of clamping mechanisms are used in combination with intravenous tubing for metering and controlling the flow of medication through the tubing and into a patient. Roller clamps are a common type of clamping mechanism used for medical purposes, particularly in combination with intravenous (IV) drip tubing. Roller clamps have a rotationally-mounted dial or rotor for selectively compressing the IV tubing in order to selectively control the fluid volume and drip-rate of medicinal fluids flowing therethrough.

Roller clamps, however, when in the fully open state, have a tendency to slide down the IV tubing. If the IV tubing is long enough to reach the floor, it is inconvenient for a medical practitioner to have to bend down to the floor level to operate the clamp, and further, the roller clamp could be exposed to the unsanitary conditions of the floor.

In the past, adhesive tape has been utilized to secure the roller clamp in position on the IV tubing. However, adhesive tape is difficult to remove from the clamp and also leaves adhesive residue on the clamp and on the tubing. The adhesive can pick up dirt and bacteria and create an unsanitary condition for the patient. Consequently, there is a need for a hygienic device that prevents a roller clamp from sliding down IV infusion tubing while maintaining the tubing in a patent condition.

Additionally, when used in conjunction with a volume pump, such as a Baxter pump, it would be desirable to have a non-residue device, in that the electronic sensors in the pump may be affected by adhesive and other residue. Further, the roller clamp must be maintained in the fully open position below the volume pump. Thus, it would be desirable to provide an intravenous tubing clamp allowing the user to maintain the position and condition of the roller clamp. For such a clamp, one would require the intravenous clamping device to grasp the intravenous tubing without impinging on the drip rate of the volume pump.

Thus, an intravenous tubing attachment solving the aforementioned problems is desired.

### SUMMARY OF THE INVENTION

The intravenous tubing attachment is a detachable device for restricting movement of a clamp mounted on intravenous tubing. The intravenous tubing attachment includes a main body portion having a pair of opposed planar surfaces, which are sized and shaped for receiving optional adhesive labels. The main body portion is formed from a hard plastic, and the opposed planar surfaces may be smooth for easy attachment and detachment of the adhesive labels, which may identify medication or a specific patient.

A pair of arcuate jaws integrally formed on a proximal end of the main body portion define a clip for releasably engaging the intravenous tubing without restricting fluid flow through the intravenous tubing. The jaws define a recess dimensioned and configured for engaging the intravenous tubing. The engagement of the intravenous tubing with the recess maintains the intravenous tubing attachment in one selectable position on the tubing and restricts movement of a clamp mounted on the intravenous tubing, preventing slippage of the clamp on the tubing.

These and other features of the present invention will become readily apparent upon further review of the following specification and drawings.

### BRIEF DESCRIPTION OF THE DRAWINGS

FIG. 1 is an environmental, perspective view of an intravenous tubing attachment according to the present invention.

FIG. 2 is a perspective view of the intravenous tubing attachment according to the present invention with an optional label exploded therefrom.

FIG. 3 is a side view of the intravenous tubing attachment according to the present invention.

Similar reference characters denote corresponding features consistently throughout the attached drawings.

### DETAILED DESCRIPTION OF THE PREFERRED EMBODIMENT

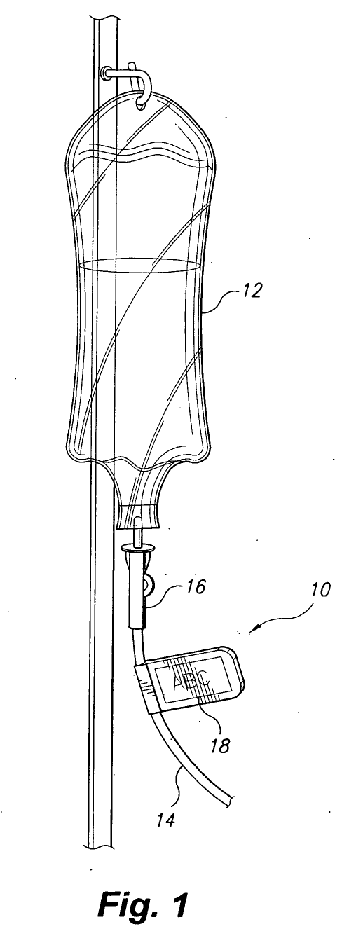

As shown in FIG. 1, the intravenous (IV) tubing attachment 10 is adapted for removable attachment to conventional IV infusion tubing 14. As will be described in further detail below, the IV tubing attachment 10 engages IV tubing 14 in such a manner that IV tubing attachment 10 maintains a stable position on the IV tubing 14 without restricting the flow of fluid through IV tubing 14.

In a conventional IV system, such as that shown in FIG. 1, medicinal fluids are stored in IV bag 12. The medicinal fluids flow by gravity through IV tubing 14 into a patient's bloodstream. The volume and rate of fluid flow is selectively controlled through the use of a clamp mounted on IV tubing 14, such as the roller clamp 16 shown in FIG. 1. Roller clamps and similar clamping mechanisms, however, can become loose and slide down the IV tubing 14, particularly when set to their fully open positions. In order to maintain proper positioning of the clamp 16 for both convenience and sanitary reasons, IV tubing attachment 10 restricts movement of clamp 16 on the IV tubing 14, creating a barrier or block to the downward fall of the clamp 16 under the force of gravity. It should be understood that clamp 16, tubing 14 and IV bag 12 are shown in FIG. 1 for exemplary purposes only, and that IV tubing attachment 10 may be used in combination with any suitable IV bag, IV tubing and clamping mechanism.

The IV tubing attachment 10 is formed of an easily cleanable, safe and hypoallergenic material, such as, for example, but not limited to, hard plastic. The size and shape of IV tubing attachment 10 are dependent upon the needs and desires of the user. In the preferred embodiment, the IV tubing attachment has a substantially rectangular contour, having a length of approximately 1½ inches, a width of approximately one inch, and a thickness of approximately ⅛ of an inch. The foregoing dimensions are representative for purposes of enablement only, and are not intended by way of limitation.

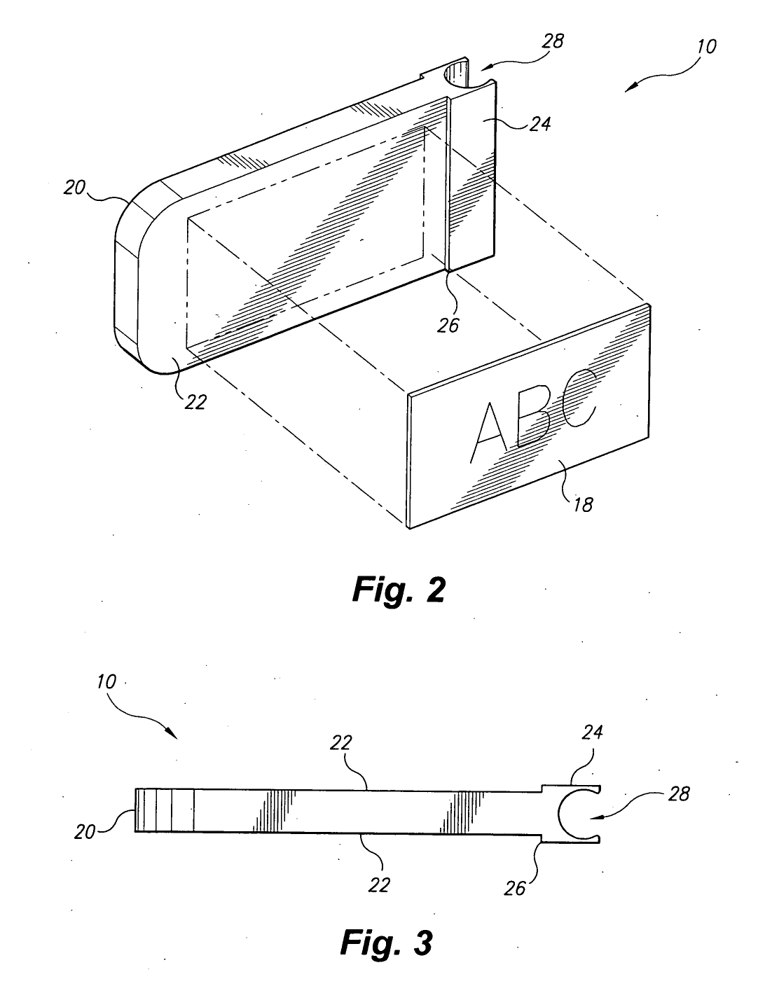

As best shown in FIG. 2, the IV tubing attachment 10 includes a main body portion 20 and a clip 24, which is integrally formed on a proximal end of the main body portion 20. The main body portion 20, in the preferred embodiment, has a substantially rectangular contour and has a pair of opposed planar surfaces 22. Each of the planar surfaces 22 is adapted for receiving an adhesive label 18, as shown.

Each of the opposed planar surfaces 22 provides a surface for the selective attachment and detachment of an adhesive label, such as label 18, shown in FIG. 2. Label 18 may indicate the type of fluid held within IV bag 12, the name of the patient, or any other suitable or necessary medical or identification information. The distal end of main body portion 20 may have rounded corners, as shown in FIG. 2, so that the IV tubing attachment 10 may be easily grasped without the risk of injury to the patient or the user.

As shown in FIGS. 2 and 3, the clip 24 is formed on the proximal end of main body portion 20. The clip 24 has a pair of arcuate jaws defining a recess 28 formed therein for receiving IV tubing 14. As best shown in FIG. 3, the clip 24 is configured so that recess 28 matches the contour of IV tubing 14. Recess 28 is contoured so that clip 24 securely grasps IV tubing 14 and holds the IV tubing attachment 10 thereto through frictional engagement therewith, but without pinching the IV tubing 14 and restricting fluid flow therethrough. As shown, the proximal ends of clip 24 are slightly tapered, allowing for easy reception of IV tubing 14 within recess 28, and allowing the IV tubing attachment 10 to be easily slid upon IV tubing 14 to selectively position the attachment 10 thereon. Preferably, the jaws define an arc slightly greater than 180° to retain the clip 24 on the tubing.

The intravenous tubing attachment 10 may be formed from substantially rigid material for frictional engagement with the tubing 14, the tubing 14 being sufficiently flexible and resilient for insertion between the jaws of the clip 24, or the jaws of the clip 24 may be slightly flexible and resilient in order to expand during insertion and removal of the tubing 14 between the jaws of the clip, but contracting after insertion of the tubing 14 in order to firmly seat the clip 24 for resilient engagement with the tubing 14, but without constricting the tubing 14, thereby maintaining the lumen in an unobstructed state so that the flow of fluid is controlled by the roller clamp 16.

Further, as best shown in the side view of FIG. 3, clip 24 has a thickness slightly greater than that of main body portion 20, allowing edge 26 of the clamping portion 24 to form a slightly raised lip, with respect to surfaces 22. This raised edge or lip provides a guide for the positioning and placement of labels 18 on opposed surfaces 22.

It is to be understood that the present invention is not limited to the embodiment described above, but encompasses any and all embodiments within the scope of the following claims.

We claim:

1. An intravenous tubing attachment, comprising:
a main body portion having a pair of opposed planar surfaces and longitudinally opposed proximal and distal ends;
a pair of arcuate jaws extending from the proximal end of the main body portion defining a recess and forming a clip dimensioned and configured for removable attachment to an intravenous infusion line below a flow-regulating infusion clamp, the clip being retained on the infusion line in order to prevent the clamp from sliding down the infusion line.

2. The intravenous tubing attachment as recited in claim 1, wherein said main body portion has a substantially rectangular contour.

3. The intravenous tubing attachment as recited in claim 2, wherein said main body portion has laterally opposed proximal and distal edges, and longitudinally opposed upper and lower edges, whereby a first corner defined by the intersection of the distal edge and the upper edge and a second corner defined by the intersection of the distal edge and the lower edge each have a substantially arcuate contour.

4. The intravenous tubing attachment as recited in claim 1, wherein said main body portion has a first thickness associated therewith, and the clip has a second thickness associated therewith, said second thickness being greater than said first thickness.

5. The intravenous tubing attachment as recited in claim 4, wherein the clip defines a pair of raised lips, each of the raised lips being formed on the proximal end of said main body portion and extending outwardly from a respective one of the planar surfaces thereof.

6. The intravenous tubing attachment as recited in claim 5, wherein each of the raised lips is sized and contoured to guide the placement of a label on a respective corresponding one of the planar surfaces.
